(19) United States
(12) Patent Application Publication
(10) Pub. No.: US 2009/0186120 A1
(43) Pub. Date: Jul. 23, 2009

(54) PROCESS TO LOSE WEIGHT, DRAIN UNWANTED BODY FATS AND OTHER TOXINS

(76) Inventor: Juanito B. Calagui, Passaic, NJ (US)

Correspondence Address: Mr. Walter J. Tencza, Jr., Suite 210, 100 Menlo Park, Edison, NJ, 08837, US

(21) Appl. No.: 12/413,881

(22) Filed: Mar. 30, 2009

Related U.S. Application Data
(63) Continuation-in-part of application No. 12/107,137, filed on Apr. 22, 2008.
Application No. 12/107,137 is a continuation-in-part of application No. 12/028,947, filed on Feb. 11, 2008.
Application No. 12/028,947 is a continuation-in-part of application No. 11/927,936, filed on Oct. 30, 2007.
Application No. 11/927,936 is a continuation-in-part of application No. 11/306,610, filed on Jan. 4, 2006, now abandoned.
Application No. 11/306,610 is a continuation-in-part of application No. 10/601,684, filed on Jun. 23, 2003, now abandoned.
Application No. 10/601,684 is a continuation-in-part of application No. 10/662,608, filed on Sep. 15, 2003, now abandoned.

(51) Int. Cl. A23F 3/40; B65D 85/72; A61P 3/04

(52) U.S. Cl. 426/2; 426/115; 426/597

(57) ABSTRACT

A method is disclosed comprising placing about thirty-two fluid ounces of water into a receptacle, placing the receptacle with water on a burner and bringing the water to a boil, ceasing to heat the water, adding about one tablespoonful or five grams of tea beverage flavorant to the water; and brewing the combination of the water and the tea beverage flavorant for about three to five minutes. The method may further include filtering out any solid tea in the combination of water and tea beverage flavorant, and consuming the combination of the water and the tea beverage flavorant.

### CROSS REFERENCE TO RELATED APPLICATIONS

The present application is a continuation in part of and claims the priority of U.S. patent application Ser. No.: 12/107,137, entitled “PROCESS TO LOSE WEIGHT, DRAIN UNWANTED BODY FATS AND OTHER TOXINS”, filed on Apr. 22, 2008, inventor and applicant Juanito B. Calagui, which is a continuation in part of and claims the priority of U.S. patent application Ser. No. 12/028,947, entitled “PROCESS TO LOSE WEIGHT, DRAIN UNWANTED BODY FATS AND OTHER TOXINS”, filed on Feb. 11, 2008, inventor and applicant Juanito B. Calagui, which is a continuation in part of and claims the priority of U.S. patent application Ser. No. 11/927,936, entitled “PROCESS TO LOSE WEIGHT, DRAIN UNWANTED BODY FATS AND OTHER TOXINS”, inventor and applicant Juanito B. Calagui, filed on Oct. 30, 2007, which is a continuation in part of and claims the priority of patent application Ser. No. 11/306,610, entitled “PROCESS TO LOSE WEIGHT, DRAIN UNWANTED BODY FATS AND OTHER TOXINS”, inventor and applicant Juanito B. Calagui, filed on Jan. 4, 2006, which is a continuation in part of and claims the priority of patent application Ser. No. 10/601,684 entitled “PROCESS TO LOSE WEIGHT, DRAIN UNWANTED BODY FATS AND OTHER TOXINS”, inventor and applicant Juanito B. Calagui, filed on Jun. 23, 2003, and patent application Ser. No. 10/662,608 entitled “PROCESS TO LOSE WEIGHT, DRAIN UNWANTED BODY FATS AND OTHER TOXINS”, inventor and applicant Juanito B. Calagui, filed on Sep. 15, 2003. The present application claims the priority of all of the above patent applications including serial nos. 12/107,137; 12/028,947; 11/927,936; 11/306,610; 10/601,684; and 10/662,608.

### FIELD OF THE INVENTION

This invention relates to improved methods and apparatus concerning losing weight.

### BACKGROUND OF THE INVENTION

It is very common practice for kids, teenagers and adults to keep eating and eating whenever foods are available. This causes them to become overweight. Overweight individuals use diet drinks, diet pills or join physical fitness centers to lose weight and to drain unwanted body fats. However, many of these options do not result in loss of weight or fat for many overweight people.

### SUMMARY OF THE INVENTION

The present invention in one or more embodiments provides a process which cleanses and dissolves foreign bodies within the human body.

A method is disclosed comprising the steps of placing about thirty-two fluid ounces of water into a receptacle, placing the receptacle with water on a burner and bringing the water to a boil, ceasing to heat the water, adding about one tablespoonful or five grams of tea beverage flavorant to the water; and brewing the combination of the water and the tea beverage flavorant for about three to five minutes. The method may further include filtering out any solid tea in the combination of water and tea beverage flavorant, and consuming the combination of the water and the tea beverage flavorant.

The temperature of the combination of the water and the tea beverage flavorant at the time that the water and the tea beverage flavorant are consumed may be between one hundred forty and one hundred sixty degrees Fahrenheit. The combination of the water and the tea beverage flavorant may be placed into a disposable plastic bottle. The combination of the water and the tea beverage flavorant may be called the mixture.

The steps of the method, including the consumption of the mixture, may be performed, twice a day, typically once after breakfast and once before dinner. The inventor has found that such a regimen results in a good overall cleansing effect and in a loss of weight.

A mixture of water and a tea beverage flavorant together in the proportions of about thirty-two fluid ounces for about every one tablespoonful or every five grams of tea beverage flavorant can be placed into a disposable plastic bottle. The mixture can be later heated and consumed.

The present invention also provides an apparatus comprising a disposable plastic bottle, which has a mixture of water and a tea beverage flavorant in the proportions of about thirty-two fluid ounces of water for about every one tablespoonful or every five grams of tea beverage flavorant, located inside the disposable plastic bottle.

### BRIEF DESCRIPTION OF THE DRAWINGS

FIG. 1 shows a flow chart of a method in accordance with an embodiment of the present invention;

FIG. 2 shows a disposable plastic bottle which includes a mixture of water and tea beverage flavorant; and

FIG. 3 shows a flow chart of a method in accordance with another embodiment of the present invention.

### DETAILED DESCRIPTION OF THE DRAWINGS

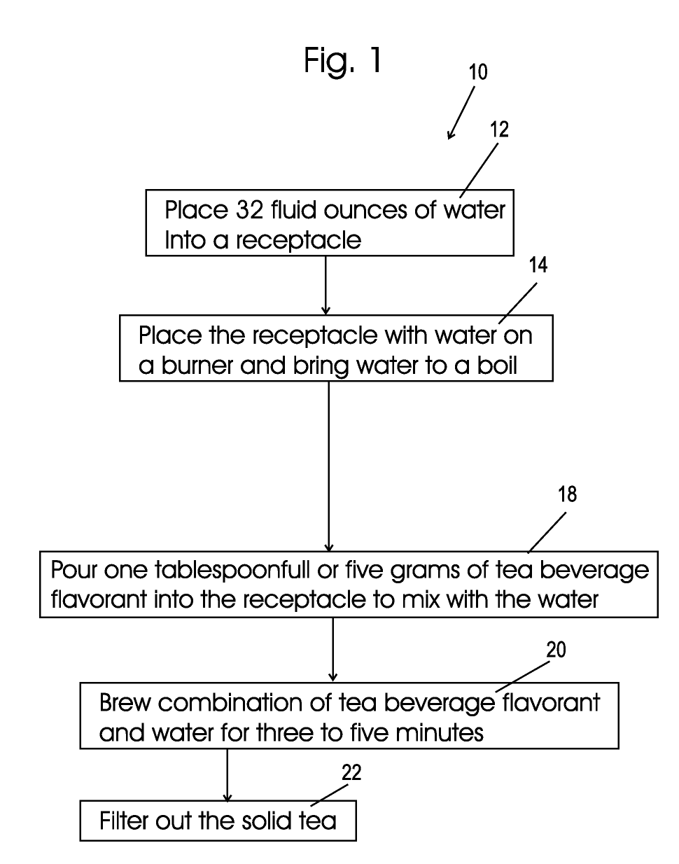

FIG. 1 shows a flow chart 10 of a method in accordance with an embodiment of the present invention. The method begins with placing thirty-two fluid ounces of water into a receptacle at step 12. The receptacle, with the water, is then placed on a burner and brought to a boil at step 14. At step 18 one tablespoonful of tea beverage flavorant or five grams is poured into the receptacle to mix with the water. At step 20 the combination of water and tea beverage flavorant is brewed for three to five minutes. At step 22 the solid tea in the combination of water and tea beverage flavorant is filtered out. The remaining combination or drink can be consumed by an individual.

The remaining drink should be at 140-160 degrees Fahrenheit when consumed by an individual for the best cleansing effect for dissolving foreign bodies and for loss of weight generally. The applicant has tested a remaining drink with the temperature between 140-160 degrees Fahrenheit versus drinks of lower temperatures and has found that the temperature range of 140-160 degrees Fahrenheit is optimum for dissolving toxins and cleansing of the body and for loss of weight.

A method in accordance with an embodiment of the present invention may include repeating the procedure shown in FIG. 1, everyday, twice a day, typically once after breakfast and once before dinner.

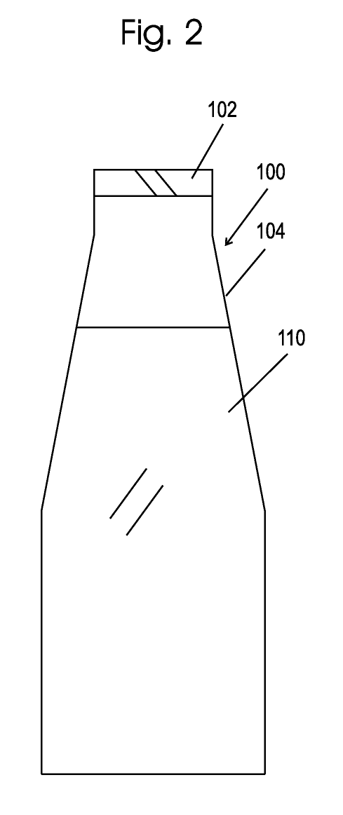

In addition, the method may additionally include placing the mixture of a ratio of 32 fluid ounces of water to one tablespoonful or five grams of tea flavorant into a disposable plastic bottle, which can be sold to a purchaser. FIG. 2 shows a disposable plastic bottle 100. The bottle 100 is transparent in this example and includes a cap 102 and a body portion 104 or receptacle for holding liquid. The body portion 104 in FIG. 2 holds a mixture of water and tea beverage flavorant 110 in the proportions of thirty-two fluid ounces of water for every one tablespoonful or every five grams of tea beverage flavorant. The cap 102 provides a seal so that the mixture 110 is held in a sealed, enclosed chamber inside the bottle 100. The mixture 110 in the disposable plastic bottle 100 can be readily heated to the appropriate temperature and consumed.

In one embodiment, draining unwanted body fat and other toxins may follow thirty-three to thirty-six days after the process has been started.

The present invention in one or more embodiments, introduces a new process to lose weight, drain unwanted body fats and other toxins to cleanse within a living structure the human body to be healthy, pain free and cancer free.

The human body is a living structure of multiple mechanical assembly links and associated to the function of each or the other assembly. The above-mentioned linkage and association of the assembly within the human body are through the blood vessels or flowing channels and within these channels malfunctions and other irregularities are mostly caused by the buildup of fats likewise, the buildup of fats will eventually develop into the blockade of the blood vessels or flowing channels and the blockade will cause the tissue fluid to lose contact within an organ to dehydrate and collapse or die and in the end decaying tissue fluid will then turn into toxin to cause cancer. The new process is to dissolve and prevent the buildup of fats within tissue fluid and the blood vessels or flowing channels of a human being and also to dissolve pockets of fats in other portions of the body of a human being. The process in accordance with one or more embodiments of the present invention act as a cleansing agent and is effective to drain unwanted body fats and other toxins to help an individual to be healthy and to enjoy a better life.

The present invention in one or more embodiments has a beneficial effect on the heart, the respiratory system, and helps individuals to be cancer free with the use of a new process in accordance with one or more embodiments of the present invention. The malfunctions and irregularities of the blood vessels are most often caused by the buildup of fats that slow down the deliveries of nutrient-rich blood and dehydrate the muscles and linkages to the organs.

One or more embodiments of the present invention can be used for medical and dental reasons and as preventive maintenance to help a human being to be pain free, cancer free, and healthy. It is also beneficial for people suffering from asthma. It is often difficult to detect why sicknesses, such as cancer and diabetes occur, and embodiments of the present invention are useful in ameliorating those sicknesses.

The mixture of tea is typically heated to a temperature of between one hundred forty and one hundred sixty degrees Fahrenheit.

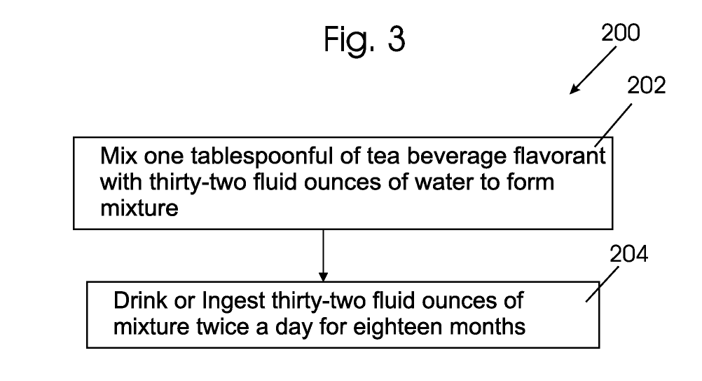

FIG. 3 shows a flow chart 200 of a method in accordance with another embodiment of the present invention. At step 202 one tablespoonful of tea beverage flavorant is mixed with about thirty-two fluid ounces of water to form a mixture and the temperature of the mixture should be between one hundred forty and one hundred sixty degrees Fahrenheit. At step 204 a human being would then drink or ingest about thirty-two fluid ounces of the mixture twice a day for eighteen months and stopped to prevent dehydration.

One or more embodiments of the present invention help prevent and stop cancer and the Tikuanyin (trademarked) tea has a cleansing agent to cleanse the blood vessels and the organs that were partly coated on the inside with fat. The build up of fat causes a wide range of damage to the organs and the depositing of fat into the organs continues during a person's entire life. A bowel movement demonstrates the draining of fat from the human body. A loose bowel movement drains first without the presence of fat. The start of the draining of fat from the human body will follow twenty-seven to thirty-six days after the initial regimen of drinking or ingesting a mixture in accordance with an embodiment of the present invention into the human body, such as the mixture of one tablespoonful of tea beverage flavorant mixed with about thirty-two fluid ounces of water, at a temperature of about one hundred forty to one hundred sixty degrees Fahrenheit.

If the user of a mixture in accordance with an embodiment of the present invention, is twenty to forty years old, the user of the mixture needs to drink or ingest the mixture for twenty months and then should stop to prevent dehydration. Individuals forty years old and older should drink the mixture for eighteen months and stopped to prevent dehydration.

The new process is the only effective procedure to drain unwanted body fats and other toxins to help an individual prevent the mechanics that cause osteoperosis, AIDS (Acquired Immune Deficiency Syndrome), epilepsy, lupus erythematosus, leukemia, gall bladder cancer, breast cancer, malaria, autism, colon cancer, and pancreatic cancer and keeping the channels leading to the reproductive organs clean, to keep the reproductive organs always ready.

The depositing of fat into the organs continues during a person's entire life. This is at least in part because, fat is always a portion of the blood pumped from the heart into the organs to feed the organs. The fat causes a wide range of damage to the organs.

Although the invention has been described by reference to particular illustrative embodiments thereof, many changes and modifications of the invention may become apparent to those skilled in the art without departing from the spirit and scope of the invention. It is therefore intended to include within this patent all such changes and modifications as may reasonably and properly be included within the scope of the present invention's contribution to the art.

I claim:

1. A method comprising the steps of:
placing about thirty-two fluid ounces of water into a receptacle;
placing the receptacle with water on a burner and bringing the water to a boil;
ceasing to heat the water;
adding about one tablespoonful of tea beverage flavorant to the water; and
brewing the combination of the water and the tea beverage flavorant for about three to five minutes.

2. The method of claim 1 further comprising
filtering out any solid tea in the combination of water and tea beverage flavorant.

3. The method of claim 1 further comprising
consuming the combination of the water and the tea beverage flavorant.

4. The method of claim 3 wherein
the temperature of the combination of the water and the tea beverage flavorant at the time that the water and the tea beverage flavorant are consumed is between one hundred forty and one hundred sixty degrees Fahrenheit.

5. The method of claim 1 further comprising
placing the combination of the water and the tea beverage flavorant into a disposable plastic bottle.

6. The method of claim 1 further comprising
performing the previous steps once after breakfast.

7. The method of claim 1 further comprising
performing the previous steps once before dinner.

8. The method of claim 1 further comprising
performing the previous steps once after breakfast and once before dinner.

9. A method comprising the steps of
mixing water and a tea beverage flavorant together in the proportions of about thirty-two fluid ounces for about every one tablespoonful of tea beverage flavorant to form a mixture;
and placing the mixture in a disposable plastic bottle.

10. A method comprising the steps of
mixing water and a tea beverage flavorant together in the proportions of about thirty-two fluid ounces for about every one tablespoonful of tea beverage flavorant to form a mixture;
and consuming the mixture.

11. An apparatus comprising
a disposable plastic bottle; and
a mixture of water and a tea beverage flavorant in the proportions of about thirty-two fluid ounces of water for about every one tablespoonful of tea beverage flavorant;
and wherein the mixture is located inside the disposable plastic bottle.

12. A method comprising the steps of:
placing about thirty-two fluid ounces of water into a receptacle;
placing the receptacle with water on a burner and bringing the water to a boil;
ceasing to heat the water;
adding about five grams of tea beverage flavorant to the water; and
brewing the combination of the water and the tea beverage flavorant for about three to five minutes.

13. The method of claim 12 further comprising
filtering out any solid tea in the combination of water and tea beverage flavorant.

14. The method of claim 12 further comprising
consuming the combination of the water and the tea beverage flavorant.

15. The method of claim 14 wherein
the temperature of the combination of the water and the tea beverage flavorant at the time that the water and the tea beverage flavorant are consumed is between one hundred forty and one hundred sixty degrees Fahrenheit.

16. The method of claim 12 further comprising
placing the combination of the water and the tea beverage flavorant into a disposable plastic bottle.

17. A method comprising the steps of
mixing water and a tea beverage flavorant together in the proportions of about thirty-two fluid ounces for about every five grams of tea beverage flavorant to form a mixture;
and placing the mixture in a disposable plastic bottle.

18. A method comprising the steps of
mixing water and a tea beverage flavorant together in the proportions of about thirty-two fluid ounces for about every five grams of tea beverage flavorant to form a mixture;
and consuming the mixture.

19. An apparatus comprising
a disposable plastic bottle; and
a mixture of water and a tea beverage flavorant in the proportions of about thirty-two fluid ounces of water for about every five grams of tea beverage flavorant;
and wherein the mixture is located inside the disposable plastic bottle.

20. A method comprising
during the drinking and ingesting of the mixture into a human body about thirty two fluid ounces of the mixture at least twice a day, including once after breakfast and once before dinner, wherein the mixture is in a ratio of about one tablespoonful of tea beverage flavorant to thirty-two fluid ounces of water;
wherein the drinking and ingesting continue for a period of about eighteen months and then stopped to prevent dehydration; and
wherein the mixture has a temperature of of approximately between one hundred forty and one hundred sixty degrees Fahrenheit.
